(19) United States
(12) Patent Application Publication
(10) Pub. No.: US 2006/0057196 A1
(43) Pub. Date: Mar. 16, 2006

(54) Efavirenz tablet formulation having unique biopharmaceutical characteristics

(76) Inventors: Munir A. Hussain, Belle Meade, NJ (US); Julia Zh. Gao, Plainsboro, NJ (US); Rajeshwar Motheram, Dayton, NJ (US); David B. Gray, Newark, DE (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/253,413

(22) Filed: Oct. 19, 2005

Related U.S. Application Data
(63) Continuation of application No. 10/304,644, filed on Nov. 26, 2002, now abandoned.
(60) Provisional application No. 60/333,651, filed on Nov. 27, 2001.
(60) Provisional application No. 60/360,395, filed on Feb. 28, 2002.

(51) Int. Cl. A61K 9/20

(52) U.S. Cl. 424/464

(57) ABSTRACT

The present invention provides an efavirenz tablet formulation which, when administered as a single dose to a subject, provides a mean maximum plasma concentration (Cmax) of about 4 μM to about 14 μM, a mean time of maximum plasma concentration (Tmax) of about 2 hours to about 5 hours, and a mean area under the plasma concentration versus time curve from time zero to time infinity (AUC) of about 190 μM [symbol] hour to about 470 μM [symbol] hour.

### RELATED APPLICATIONS

This application claims benefit to provisional applications U.S. Ser. No. 60/333,651 filed Nov. 27, 2001 and U.S. Ser. No. 60/360,395 filed Feb. 28, 2002. The entire teachings of the referenced applications are incorporated herein by reference.

### FIELD OF THE INVENTION

The present invention is directed to novel efavirenz tablet formulations having unique biopharmaceutical characteristics which are useful for treating human immunodeficiency virus type-1 (HIV-1) infection, and methods of treating HIV-1 infection employing such compositions.

### BACKGROUND OF THE INVENTION

Efavirenz, (s)6-chloro-4-(cyclopropylethynyl)-1,4-dihydro-4-(trifluoromethyl)-2H-3,1-benzoxazin-2-one, is a non-nucleoside inhibitor of HIV-1 reverse transcriptase (NNRTI), and may be used in combination with other anti-retroviral agents for the treatment of HIV-1 infection in children and adults. The active ingredient of the tablet formulation of the present invention is the NNRTI efavirenz, which is present in a therapeutically effective amount. Methods for the synthesis of efavirenz are disclosed in U.S. Pat. Nos. 5,519,021, 5,663,169, 5,665,720 and 5,811,423. The disclosure of U.S. Pat. Nos. 5,519,021, 5,663,169, 5,665,720 and 5,811,423 in their entirety are hereby incorporated by reference.

Currently, efavirenz is marketed in 50, 100 and 200 mg strength hard gelatin capsules. With a usual adult daily dose of 600 mg, the current capsule dosage form requires patients to administer multiple dosage units. In order to reduce pill burden and to aid in improving patient adherence, efavirenz tablets have been developed in strengths of 300 mg and 600 mg which have unique biopharmaceutical characteristics.

### SUMMARY OF THE INVENTION

The present invention provides an efavirenz tablet dosage form, said efavirenz tablet dosage form providing, when administered as a single dose to a subject, a mean maximum plasma concentration (Cmax) of about 4 μM to about 14 μM, a mean time of maximum plasma concentration (Tmax) of about 2 hours to about 5 hours, and a mean area under the plasma concentration versus time curve from time zero to time infinity (AUC) of about 190 μM [symbol] hour to about 470 μM [symbol] hour.

The present invention further provides a tablet dosage form having a mean area under the plasma concentration versus time curve from time zero to the last quantifiable concentration-time point (AUCT) of about 180 μM [symbol] hour to about 430 μM [symbol] hour. In one embodiment, the mean AUCT is about 270 μM [symbol] hour to about 350 μM [symbol] hour.

The present invention also provides a tablet dosage form having a mean terminal disposition half-life (T_{1/2}) of about 30 hours to about 140 hours. In one embodiment the mean T_{1/2} is about 70 hours to about 100 hours.

The present invention further provides an efavirenz tablet dosage form, said efavirenz tablet dosage form providing, when administered as a single dose to a subject, a mean maximum plasma concentration (Cmax) of about 7 μM to about 9 μM, a mean time of maximum plasma concentration (Tmax) of about 2 hours to about 5 hours, and a mean area under the plasma concentration versus time curve from time zero to time infinity (AUC) of about 250 μM [symbol] hour to about 400 μM [symbol] hour.

The present invention also provides a tablet dosage form comprising a therapeutically effective amount of efavirenz and about 4% by weight of a disintegrant relative to the total dry weight of the tablet dosage form.

The present invention further provides a kit comprising packaging including one or more efavirenz tablets and a package insert or label indicating to a user that the efavirenz tablet may be suitable for the treatment of human immunodeficiency virus Type 1 (HIV-1) infection.

The present invention provides a kit comprising at least one efavirenz tablet and a package insert or label instructing the user on dosage and administration of the tablet dosage form.

The present invention also provides a kit comprising at least one efavirenz tablet and a package insert or label warning the user of potential side effects, adverse reactions or drug interactions.

The present invention also provides a method of treating human immunodeficiency virus Type 1 (HIV-1) infection comprising administering to a mammal said efavirenz tablet formulation.

The present invention also provides a method of treating human immunodeficiency virus Type 1 (HIV-1) infection comprising administering to a mammal an efavirenz tablet dosage form, said efavirenz tablet dosage form providing, when administered as a single dose to a subject, a mean maximum plasma concentration (Cmax) of about 4 μM to about 14 μM, a mean time of maximum plasma concentration (Tmax) of about 2 hours to about 5 hours, and a mean area under the plasma concentration versus time curve from time zero to time infinity (AUC) of about 190 μM [symbol] hour to about 470 μM [symbol] hour.

In one embodiment, the present invention also provides a 300 mg efavirenz tablet dosage form suitable for use in treating human immunodeficiency virus Type 1 (HIV-1) infection, said 300 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 62% and about 75% of the efavirenz tablet dosage form is dissolved after about 10 minutes in the type II dissolution apparatus, (b) between about 90% and about 95% of the efavirenz tablet dosage form is dissolved after about 20 minutes in the type II dissolution apparatus, (c) between about 96% and about 98% of the efavirenz tablet dosage form is dissolved after about 30 minutes in the type II dissolution apparatus, and (d) between about 99% and about 100% of the efavirenz tablet dosage form is dissolved after about 45 minutes in the type II dissolution apparatus.

In another embodiment, the present invention also provides a 300 mg efavirenz tablet dosage form suitable for use in treating human immunodeficiency virus Type 1 (HIV-1) infection, said 300 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 62% and about 75% of the efavirenz tablet dosage form is dissolved after about 10 minutes in the type II dissolution apparatus, (b) between about 89% and about 95% of the efavirenz tablet dosage form is dissolved after about 20 minutes in the type II dissolution apparatus, (c) between about 94% and about 98% of the efavirenz tablet dosage form is dissolved after about 30 minutes in the type II dissolution apparatus, and (d) between about 97% and about 100% of the efavirenz tablet dosage form is dissolved after about 45 minutes in the type II dissolution apparatus.

The present invention also provides a 300 mg efavirenz tablet dosage form wherein 100% of the 300 mg efavirenz tablet dosage form is dissolved after about 45 minutes.

In one embodiment, the present invention also provides a 600 mg efavirenz tablet dosage form suitable for use in treating human immunodeficiency virus Type 1 (HIV-1) infection, said 600 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 49% and about 71% of the efavirenz tablet dosage form is dissolved after about 10 minutes in the type II dissolution apparatus, (b) between about 87% and about 95% of the efavirenz tablet dosage form is dissolved after about 20 minutes in the type II dissolution apparatus, (c) between about 97% and about 99% of the efavirenz tablet dosage form is dissolved after about 30 minutes in the type II dissolution apparatus, and (d) between about 99% and about 100% of the efavirenz tablet dosage form is dissolved after about 45 minutes in the type II dissolution apparatus.

In another embodiment, the present invention also provides a 600 mg efavirenz tablet dosage form suitable for use in treating human immunodeficiency virus Type 1 (HIV-1) infection, said 600 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 49% and about 71% of the efavirenz tablet dosage form is dissolved after about 10 minutes in the type II dissolution apparatus, (b) between about 86% and about 95% of the efavirenz tablet dosage form is dissolved after about 20 minutes in the type II dissolution apparatus, (c) between about 94% and about 99% of the efavirenz tablet dosage form is dissolved after about 30 minutes in the type II dissolution apparatus, and (d) between about 97% and about 100% of the efavirenz tablet dosage form is dissolved after about 45 minutes in the type II dissolution apparatus.

The present invention also provides a 600 mg efavirenz tablet dosage form wherein 100% of the 600 mg efavirenz tablet dosage form is dissolved after about 45 minutes.

The present invention further provides a method of treating human immunodeficiency virus Type 1 (HIV-1) infection comprising administering to a mammal a 300 mg efavirenz tablet dosage form, said 300 mg efavirenz tablet dosage form providing, an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 62% and about 75% of the efavirenz tablet dosage form is dissolved after about 10 minutes in the type II dissolution apparatus, (b) between about 90% and about 95% of the efavirenz tablet dosage form is dissolved after about 20 minutes in the type II dissolution apparatus, (c) between about 96% and about 98% of the efavirenz tablet dosage form is dissolved after about 30 minutes in the type II dissolution apparatus, and (d) between about 99% and about 100% of the efavirenz tablet dosage form is dissolved after about 45 minutes in the type II dissolution apparatus. In another embodiment, the present invention further provides a method of treating human immunodeficiency virus Type 1 (HIV-1) infection comprising administering to a mammal a 300 mg efavirenz tablet dosage form, said 300 mg efavirenz tablet dosage form providing, an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 62% and about 75% of the efavirenz tablet dosage form is dissolved after about 10 minutes in the type II dissolution apparatus, (b) between about 89% and about 95% of the efavirenz tablet dosage form is dissolved after about 20 minutes in the type II dissolution apparatus, (c) between about 94% and about 98% of the efavirenz tablet dosage form is dissolved after about 30 minutes in the type II dissolution apparatus, and (d) between about 97% and about 100% of the efavirenz tablet dosage form is dissolved after about 45 minutes in the type II dissolution apparatus.

The present invention further provides a method of treating human immunodeficiency virus Type 1 (HIV-1) infection comprising administering to a mammal a 600 mg efavirenz tablet dosage form, said 600 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 49% and about 71% of the efavirenz tablet dosage form is dissolved after about 10 minutes in the type II dissolution apparatus, (b) between about 87% and about 95% of the efavirenz tablet dosage form is dissolved after about 20 minutes in the type II dissolution apparatus, (c) between about 97% and about 99% of the efavirenz tablet dosage form is dissolved after about 30 minutes in the type II dissolution apparatus, and (d) between about 99% and about 100% of the efavirenz tablet dosage form is dissolved after about 45 minutes in the type II dissolution apparatus. In another embodiment, the present invention further provides a method of treating human immunodeficiency virus Type 1 (HIV-1) infection comprising administering to a mammal a 600 mg efavirenz tablet dosage form, said 600 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 49% and about 71% of the efavirenz tablet dosage form is dissolved after about 10 minutes in the type II dissolution apparatus, (b) between about 86% and about 95% of the efavirenz tablet dosage form is dissolved after about 20 minutes in the type II dissolution apparatus, (c) between about 94% and about 99% of the efavirenz tablet dosage form is dissolved after about 30 minutes in the type II dissolution apparatus, and (d) between about 97% and about 100% of the efavirenz tablet dosage form is dissolved after about 45 minutes in the type II dissolution apparatus.

### BRIEF DESCRIPTION OF THE FIGURES

The following figures are illustrative of embodiments of the invention and are not meant to limit the scope of the invention as encompassed by the claims.

FIG. 1 shows a process flow diagram illustrating the efavirenz tablet manufacturing procedure.

FIG. 2 shows the mean efavirenz plasma concentration versus time curves after administration to subjects in a single dose with a 600 mg total dose of tablet dosage containing 4% croscarmellose sodium (1×600 mg and 2×300 mg) and the commercial efavirenz capsule (3×200 mg) formulation; (0-504 hours).

FIG. 3 shows the mean efavirenz plasma concentration versus time curves after administration to subjects in a single dose with a 600 mg total dose of the tablet dosage containing 4% croscarmellose sodium (1×600 mg and 2×300 mg) and the commercial efavirenz capsule (3×200 mg) Formulation; (0-48 hours).

### DETAILED DESCRIPTION OF THE INVENTION

The present invention provides novel oral tablet dosage formulations (also referred to herein as dosage forms) of efavirenz that are useful in the inhibition of human immunodeficiency virus type-1 (HIV-1), the prevention or treatment of infection by HIV-1, and in the treatment (including prevention) of the resulting acquired immune deficiency syndrome (AIDS). In particular, the present invention relates to compressed tablets comprising efavirenz that have unique biopharmaceutical characteristics. The present invention also provides methods of making such tablets.

The active ingredient of the tablet dosage forms of the present invention is the NNRTI efavirenz, (s)6-chloro-4-(cyclopropylethynyl)-1,4-dihydro-4-(trifluoromethyl)-2H-3,1-benzoxazin-2-one, which is present in a therapeutically effective amount. Methods for the synthesis of efavirenz are disclosed in U.S. Pat. Nos. 5,519,021, 5,663,169, 5,665,720 and 5,811,423. The disclosure of U.S. Pat. Nos. 5,519,021, 5,663,169, 5,665,720 and 5,811,423 in their entirety are hereby incorporated by reference.

The present invention provides an efavirenz tablet dosage form, said efavirenz tablet dosage form providing, when administered as a single dose to a subject, a mean maximum plasma concentration (Cmax) of about 4 μM to about 14 μM, a mean time of maximum plasma concentration (Tmax) of about 2 hours to about 5 hours, and a mean area under the plasma concentration versus time curve from time zero to time infinity (AUC) of about 190 μM [symbol] hour to about 470 μM [symbol] hour.

The present invention further provides a tablet dosage form having a mean area under the plasma concentration versus time curve from time zero to the last quantifiable concentration-time point (AUCT) of about 180 μM [symbol] hour to about 430 μM [symbol] hour. In one embodiment, the mean AUCT is about 270 μM [symbol] hour to about 350 μM [symbol] hour.

The present invention also provides a tablet dosage form having a mean terminal disposition half-life (T_{1/2}) of about 30 hours to about 140 hours. In one embodiment, the mean T_{1/2} is about 70 hours to about 100 hours.

The present invention further provides an efavirenz tablet dosage form, said efavirenz tablet dosage form providing, when administered as a single dose to a subject, a mean maximum plasma concentration (Cmax) of about 7 μM to about 9 μM, a mean time of maximum plasma concentration (Tmax) of about 2 hours to about 5 hours, and a mean area under the plasma concentration versus time curve from time zero to time infinity (AUC) of about 250 μM [symbol] hour to about 400 μM [symbol] hour.

The present invention also provides a tablet dosage form comprising a therapeutically effective amount of efavirenz and about 4% by weight of a disintegrant relative to the total dry weight of the tablet dosage form.

The present invention further provides a kit comprising packaging including one or more efavirenz tablets and a package insert or label indicating to a user that the efavirenz tablet may be suitable for the treatment of human immunodeficiency virus Type 1 (HIV-1) infection.

The present invention provides a kit comprising at least one efavirenz tablet and a package insert or label instructing the user on dosage and administration of the tablet dosage form.

The present invention also provides a kit comprising at least one efavirenz tablet and a package insert or label warning the user of potential side effects, adverse reactions or drug interactions.

The present invention also provides a method of treating human immunodeficiency virus Type 1 (HIV-1) infection comprising administering to a mammal said efavirenz tablet formulation.

The present invention also provides a method of treating human immunodeficiency virus Type 1 (HIV-1) infection comprising administering to a mammal an efavirenz tablet dosage form, said efavirenz tablet dosage form providing, when administered as a single dose to a subject, a mean maximum plasma concentration (Cmax) of about 4 μM to about 14 μM, a mean time of maximum plasma concentration (Tmax) of about 2 hours to about 5 hours, and a mean area under the plasma concentration versus time curve from time zero to time infinity (AUC) of about 190 μM [symbol] hour to about 470 μM [symbol] hour.

The present invention also provides a 300 mg efavirenz tablet dosage form suitable for use in treating human immunodeficiency virus Type 1 (HIV-1) infection, said 300 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 62% and about 75% of the efavirenz tablet dosage form is dissolved after about 10 minutes in the type II dissolution apparatus, (b) between about 90% and about 95% of the efavirenz tablet dosage form is dissolved after about 20 minutes in the type II dissolution apparatus, (c) between about 96% and about 98% of the efavirenz tablet dosage form is dissolved after about 30 minutes in the type II dissolution apparatus, and (d) between about 99% and about 100% of the efavirenz tablet dosage form is dissolved after about 45 minutes in the type II dissolution apparatus. Furthermore, the present invention also provides a 300 mg efavirenz tablet dosage form suitable for use in treating human immunodeficiency virus Type 1 (HIV-1) infection, said 300 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 62% and about 75% of the efavirenz tablet dosage form is dissolved after about 10 minutes in the type II dissolution apparatus, (b) between about 89% and about 95% of the efavirenz tablet dosage form is dissolved after about 20 minutes in the type II dissolution apparatus, (c) between about 94% and about 98% of the efavirenz tablet dosage form is dissolved after about 30 minutes in the type II dissolution apparatus, and (d) between about 97% and about 100% of the efavirenz tablet dosage form is dissolved after about 45 minutes in the type II dissolution apparatus.

The present invention also provides a 300 mg efavirenz tablet dosage form wherein 100% of the 300 mg efavirenz tablet dosage form is dissolved after about 45 minutes.

The present invention also provides a 600 mg efavirenz tablet dosage form suitable for use in treating human immunodeficiency virus Type 1 (HIV-1) infection, said 600 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 49% and about 71% of the efavirenz tablet dosage form is dissolved after about 10 minutes in the type II dissolution apparatus, (b) between about 87% and about 95% of the efavirenz tablet dosage form is dissolved after about 20 minutes in the type II dissolution apparatus, (c) between about 97% and about 99% of the efavirenz tablet dosage form is dissolved after about 30 minutes in the type II dissolution apparatus, and (d) between about 99% and about 100% of the efavirenz tablet dosage form is dissolved after about 45 minutes in the type II dissolution apparatus. Furthermore, the present invention also provides a 600 mg efavirenz tablet dosage form suitable for use in treating human immunodeficiency virus Type 1 (HIV-1) infection, said 600 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 49% and about 71% of the efavirenz tablet dosage form is dissolved after about 10 minutes in the type II dissolution apparatus, (b) between about 86% and about 95% of the efavirenz tablet dosage form is dissolved after about 20 minutes in the type II dissolution apparatus, (c) between about 94% and about 99% of the efavirenz tablet dosage form is dissolved after about 30 minutes in the type II dissolution apparatus, and (d) between about 97% and about 100% of the efavirenz tablet dosage form is dissolved after about 45 minutes in the type II dissolution apparatus.

The present invention also provides a 600 mg efavirenz tablet dosage form wherein 100% of the 600 mg efavirenz tablet dosage form is dissolved after about 45 minutes.

The present invention further provides a method of treating human immunodeficiency virus Type 1 (HIV-1) infection comprising administering to a mammal a 300 mg efavirenz tablet dosage form, said 300 mg efavirenz tablet dosage form providing, an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 62% and about 75% of the efavirenz tablet dosage form is dissolved after about 10 minutes in the type II dissolution apparatus, (b) between about 90% and about 95% of the efavirenz tablet dosage form is dissolved after about 20 minutes in the type II dissolution apparatus, (c) between about 96% and about 98% of the efavirenz tablet dosage form is dissolved after about 30 minutes in the type II dissolution apparatus, and (d) between about 99% and about 100% of the efavirenz tablet dosage form is dissolved after about 45 minutes in the type II dissolution apparatus. Furthermore, the present invention further provides a method of treating human immunodeficiency virus Type 1 (HIV-1) infection comprising administering to a mammal a 300 mg efavirenz tablet dosage form, said 300 mg efavirenz tablet dosage form providing, an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 62% and about 75% of the efavirenz tablet dosage form is dissolved after about 10 minutes in the type II dissolution apparatus, (b) between about 89% and about 95% of the efavirenz tablet dosage form is dissolved after about 20 minutes in the type II dissolution apparatus, (c) between about 94% and about 98% of the efavirenz tablet dosage form is dissolved after about 30 minutes in the type II dissolution apparatus, and (d) between about 97% and about 100% of the efavirenz tablet dosage form is dissolved after about 45 minutes in the type II dissolution apparatus.

The present invention further provides a method of treating human immunodeficiency virus Type 1 (HIV-1) infection comprising administering to a mammal a 600 mg efavirenz tablet dosage form, said 600 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 49% and about 71% of the efavirenz tablet dosage form is dissolved after about 10 minutes in the type II dissolution apparatus, (b) between about 87% and about 95% of the efavirenz tablet dosage form is dissolved after about 20 minutes in the type II dissolution apparatus, (c) between about 97% and about 99% of the efavirenz tablet dosage form is dissolved after about 30 minutes in the type II dissolution apparatus, and (d) between about 99% and about 100% of the efavirenz tablet dosage form is dissolved after about 45 minutes in the type II dissolution apparatus. Furthermore, the present invention further provides a method of treating human immunodeficiency virus Type 1 (HIV-1) infection comprising administering to a mammal a 600 mg efavirenz tablet dosage form, said 600 mg efavirenz tablet dosage form providing an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows: (a) between about 49% and about 71% of the efavirenz tablet dosage form is dissolved after about 10 minutes in the type II dissolution apparatus, (b) between about 86% and about 95% of the efavirenz tablet dosage form is dissolved after about 20 minutes in the type II dissolution apparatus, (c) between about 94% and about 99% of the efavirenz tablet dosage form is dissolved after about 30 minutes in the type II dissolution apparatus, and (d) between about 97% and about 100% of the efavirenz tablet dosage form is dissolved after about 45 minutes in the type II dissolution apparatus.

The efavirenz tablet dosage forms of the present invention are preferably bioequivalent to the commercially available Sustiva® capsule dosage form.

Tablets of the present invention may, for example, be comprised of a predetermined amount of efavirenz (active NNRTI), croscarmellose sodium (disintegrant), microcrystalline cellulose (binder/disintegrant), sodium lauryl sulfate (surfactant), hydroxypropyl cellulose (binder), lactose monohydrate (diluent) and magnesium stearate (lubricant). The tablets have a film coating comprising, for example, Opadry® White or Yellow to visually distinguish between higher and lower dosage, and Opadry® Clear. The tablets are then preferably coated, for example, with carnuaba wax, and the dosage is printed thereon.

In one embodiment of the present invention, shown in Table 1 below, the tablet is comprised of about 300 mg efavirenz, about 24 mg croscarmellose sodium, about 120 mg microcrystalline cellulose, about 6 mg sodium lauryl sulfate, about 19.2 mg hydroxypropyl cellulose, about 124.8 mg lactose monohydrate and about 6 mg magnesium stearate. The tablet is then coated with about 18 mg Opadry® White, about 3 mg Opadry® Clear and polished with about 0.06 mg carnauba wax.

The present invention includes any efavirenz tablet formulations which have the desirable properties as set forth above.

Efavirenz tablet dosage forms as set forth above may, for example, be prepared using the specific formulations and methods described further below.

TABLE 1
Efavirenz Tablet Formulation (300 mg Tablet)

| Ingredient | Amount (mg/Tablet) |
|---|---|
| Efavirenz | 300.0 |
| Croscarmellose Sodium | 24.0 |
| Microcrystalline Cellulose | 120.0 |
| Sodium Lauryl Sulfate | 6.0 |
| Hydroxypropyl Cellulose | 19.2 |
| Lactose, Monohydrate | 124.8 |
| Magnesium Stearate | 6.0 |
| Opadry ® White | 18.0 |
| Opadry ® Clear | 3.0 |
| Carnauba Wax | 0.06 |
| Ink, Opacode ® WB, Purple | 0.03 |
| Purified Water | * |

* Removed during drying and coating processes.

In another embodiment, shown in Table 2 below, the tablet is comprised of about 600 mg efavirenz, about 48 mg croscarmellose sodium, about 240 mg microcrystalline cellulose, about 12 mg sodium lauryl sulfate, about 38.4 mg hydroxypropyl cellulose, about 249.6 mg lactose monohydrate and about 12 mg magnesium stearate. The tablet is then coated with about 24 mg Opadry® Yellow, about 6 mg Opadry® Clear and polished with about 0.12 mg carnauba wax.

TABLE 2
Efavirenz Tablet Formulation (600 mg Tablet)

| Ingredient | Amount (mg/Tablet) |
|---|---|
| Efavirenz | 600.0 |
| Croscarmellose Sodium | 48.0 |
| Microcrystalline Cellulose | 240.0 |
| Sodium Lauryl Sulfate | 12.0 |
| Hydroxypropyl Cellulose | 38.4 |
| Lactose, Monohydrate | 249.6 |
| Magnesium Stearate | 12.0 |
| Opadry ® Yellow | 24.0 |
| Opadry ®, Clear | 6.0 |
| Carnauba Wax | 0.12 |
| Ink, Opacode ® WB, Purple | 0.03 |
| Purified Water | * |

* Removed during drying and coating processes.

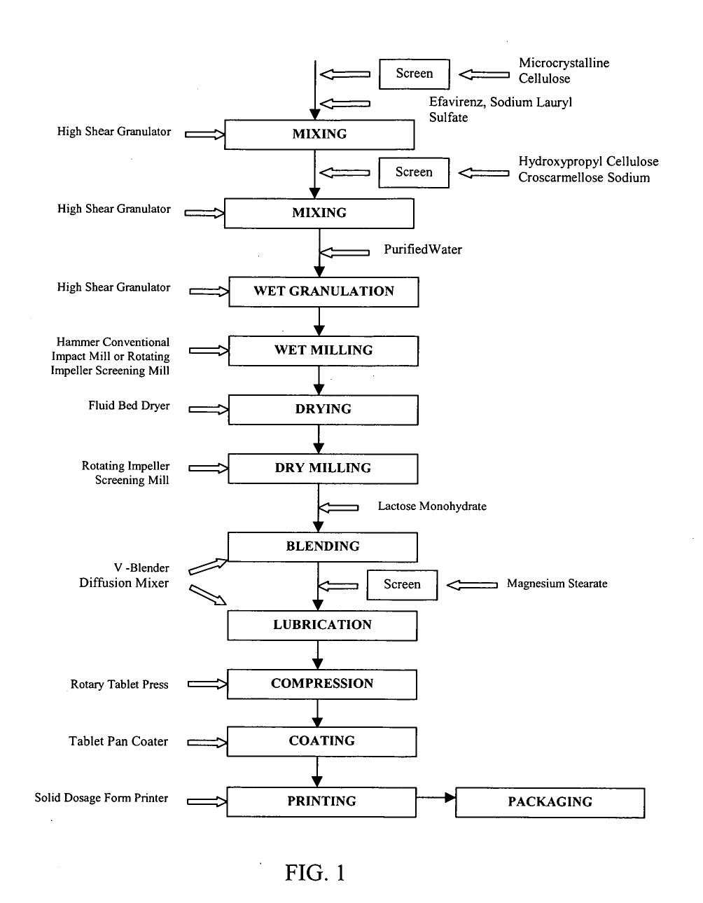

The 300 mg and 600 mg efavirenz tablet formulations of Table 1 and 2 herein may be manufactured as described below. The efavirenz, microcrystalline cellulose, sodium lauryl sulfate, croscarmellose sodium, and hydroxypropyl cellulose are first granulated using water. The granulation is then dried, milled and blended with lactose and magnesium stearate. The lubricated granulation is compressed into 300 mg strength tablets that are then coated with Opadry® White and Opadry® Clear. The 600 mg strength tablets are coated with Opadry® Yellow and Opadry® Clear. The film-coated tablets are then polished with carnuba wax for the final printing using water-based ink. A flow diagram for the manufacturing process of the two efavirenz tablet strengths described above is identical to the point of compression and is illustrated in FIG. 1. The 300 mg and 600 mg efavirenz tablets are obtained by compressing the requisite quantity of tablet blend into the appropriate size tablet. The different strength tablets are distinguished by their size and film-coating color, i.e., Opadry® White (300 mgs tablets) or Opadry® Yellow (600 mg tablets).

The individual tablet ingredients are identified and weighed according to the strength of tablet to be prepared. Microcrystalline cellulose, hydroxypropyl cellulose and croscarmellose sodium are seived as necessary. Next, microcrystalline cellulose, efavirenz and sodium lauryl sulfate are added to a conventional high shear granulator bowl. The contents are mixed for about two minutes with the mixer speed set to about 88-108 rpm and the chopper speed set to about 1242-1518 rpm.

Thereafter, hydroxypropyl cellulose and croscarmellose sodium are added to the granulator bowl. The contents are mixed for about three minutes at the same low speed setting. The contents are then granulated by adding purified water to the granulator bowl to a target power consumption reading of 11-13 kW or torque equivalent. The water spray rate is about 6±2 kg/min, and the mixer speed is about 126-154 rpm while the chopper speed is set to about 2484-3036 rpm. The mixing duration is that amount of time which is sufficient to add fluid followed by about 0-2 minutes wet massing.

The contents of the granulator bowl are then wet milled or delumped using a suitable mill (e.g., Granumill or Quadro Comil) and transferred into a fluid bed dryer bowl (e.g., Aeromatic Fluid Bed Dryer) which is preheated to about 60° C.±5° C. The contents of the dryer bowl are dried to a final loss on drying of less than or equal to about ≦2.0% w/w. The inlet air temperature is about 60° C.±5° C. (temperature excursions of about ±10° C. from the inlet temperature set point can be expected during the first 5 minutes of the drying process). The air flow rate is that amount of time which is sufficient to fluidize the bed. The dried granulation is then discharged into clean, dry polyethylene-lined containers or suitable stainless steel containers.

A rotating impeller screening mill (e.g., Quadro Comil) is then set up with a round type 0.045″ screen and standard impeller with a gap of less than 0.025″ between the impeller and the screen. The rotor speed is set such that the average velocity between the impeller and the screen is between about 2-6 meters per second. The following ingredients are then added in order to a 30 cubic foot V-Blender, diffusion mixer (e.g., Patterson Kelley) through the rotating impeller screening mill, a portion of the dried granulation, lactose monohydrate and the remainder of the dried granulation. The ingredients are then mixed for about 18 minutes.

Magnesium stearate is then screened through a US#30 mesh screen into a clean, dry polyethylene-lined drum or suitable stainless steel container. The magnesium stearate is then added to the contents of the 30 cubic foot V-Blender, diffusion mixer. The ingredients are then mixed for about 5 minutes and then discharged into polyethylene-lined drums or suitable stainless steel containers as the final tablet blend. The tablet blend is then compressed using a rotary tablet press (e.g., Courtoy R/100) to prepare 300 mg and 600 mg tablets. The tablets are passed through a tablet deduster and placed in clean, dry fiber or plastic or suitable stainless steel containers double lined with polyethylene bags.

The 300 mg strength tablets are then coated using Opadry® White dispersion and the 600 mg strength tablets are coated using Opadry® Yellow dispersion to an approximate weight gain of about 3.0% and about 2% respectively. The exhaust temperature is about 44-50° C. and the inlet temperature is adjusted to maintain the exhaust temperature.

When the weight gain of sampled tablets is achieved, the spray and inlet air heat is turned off. The tablets are then coated with Opadry® Clear until an approximate weight gain of about 0.5% is achieved for both the 300 mg and 600 mg strength tablets.

The finished tablets provide excellent content uniformity because efavirenz comprises a relatively high proportion of the formulation.

The efavirenz tablets may be subjected to in vitro dissolution studies according to U.S. Pharmacopeia XXIV (USP XXIV) procedure <711> to determine compliance with dissolution requirements.

Two batches of the 300 mg and 600 mg efavirenz dosage forms of the present invention were tested using a suitable dissolution apparatus with a rotating paddle such as “Apparatus 2” as defined by USP XXIV procedure <711>. The paddle speed was set to 50 rpm and the dissolution medium was 2.0% (w/v) aqueous sodium lauryl sulfate at 37° C. The wire sinkers used were 316 stainless steel wire, 0.032 inch/20 gauge. Absorbance was determined using a suitable single or dual-beam UV spectrophotometer at a wavelength of 247±2 nm using a 1-cm pathlength and solvent as the reference. Samples were taken at 10, 20, 30, 45 and 60 minutes. The results of the two batches of the 300 mg and 600 mg efavirenz dosage forms are listed below in Tables 3A and 3B.

TABLE 3A
In Vitro dissolution data for 300 mg and 600 mg
efavirenz dosage forms of the present invention.

| | % Dissolved | % Dissolved |
|---|---|---|
| Time | for | for |
| (minutes) | 300 mg Tablet | 600 mg Tablet |
| 0 | 0 | 0 |
| 10 | 62-75 | 49-71 |
| 20 | 90-95 | 87-95 |
| 30 | 96-98 | 97-99 |
| 45 | 99-100 | 99-100 |
| 60 | 100-101 | 100-101 |

TABLE 3B
In Vitro dissolution data for 300 mg and 600 mg
efavirenz dosage forms of the present invention.

| | % Dissolved | % Dissolved |
|---|---|---|
| Time | for | for |
| (minutes) | 300 mg Tablet | 600 mg Tablet |
| 0 | 0 | 0 |
| 10 | 62-75 | 49-71 |
| 20 | 89-95 | 86-95 |
| 30 | 94-98 | 94-99 |
| 45 | 97-100 | 97-100 |
| 60 | 99-101 | 98-101 |

The finished efavirenz tablets of the present invention are administered to individuals/study groups in order to evaluate the pharmacokinetics of the efavirenz tablets. The following pharmacokinetic parameters for efavirenz are assessed following a single dose administration of the efavirenz tablet dosage form to a subject: Cmax, Tmax, AUC, AUCT, λn, t½ and Clo.

Cmax is defined as the observed maximum plasma concentration. Tmax is defined as the time of observed maximum plasma concentration. AUCT is defined as the area under the plasma concentration-time curve from time zero to the last quantifiable concentration-time point, calculated by linear trapezoidal rule. AUC is defined as the area under the plasma concentration-time curve from time zero to time infinity; calculated as AUCT+Clast/λn, where the Clast is the last quantifiable concentration. λn is defined as the terminal or disposition rate constant, calculated as the negative slope (by linear regression) of the terminal natural log (ln)-linear portion of the plasma concentration-time curve. t½ is defined as the terminal disposition half-life; calculated as 0.693/λn. Clo is defined as the apparent oral clearance; calculated as dose/AUC.

The tablet formulations of the present invention are preferably bioequivalent to the commercial Sustiva® capsule dosage forms.

The bioequivalence studies are performed as single dose bioequivalency studies of the tablet formulations of the present invention in comparison to the commercial Sustiva® capsule formulation. The results of such a bioequivalence study are shown in Tables 4 and 5 below.

TABLE 4
Pharmacokinetic Parameters for Subjects Administered as a Single
Dose 600 mg Efavirenz Doses of the Tablet Formulations (as
described in Table 1 and 2 and further described herein)
Containing 4% Croscarmellose Sodium (2 × 300 mg and
1 × 600 mg) and Commercial Capsule Formulation (3 × 200 mg)
of Efavirenz.

| | | | 600 mg | 200 mg |
|---|---|---|---|---|
| | | 300 mg Tablet | Tablet | Capsule |
| | | 2 × 300 mg | 1 × 600 mg | 3 × 200 mg |
| Pharmacokinetic | | (Test) | (Test) | (Reference) |
| Parameter | | N = 21 | N = 21 | N = 21 |
| Cmax, μM | Mean | 7.62 | 8.06 | 7.50 |
| | SD | 2.26 | 1.95 | 2.81 |
| Tmax, h | Median | 3.0 | 4.0 | 4.0 |
| | Range | 2.0-5.0 | 2.0-8.0 | 2.0-5.0 |
| AUCT, μM · h | Mean | 332.57 | 338.77 | 326.97 |
| | SD | 116.92 | 111.37 | 112.47 |
| AUC, μM · h | Mean | 363.28 | 373.24 | 359.01 |
| | SD | 124.75 | 121.73 | 118.56 |
| λn, h⁻¹ | Mean | 0.0091 | 0.0089 | 0.0091 |
| | SD | 0.0034 | 0.0031 | 0.0035 |
| t½, h | Mean^{a} | 76.03 | 78.21 | 75.81 |
| | SD^{a} | 28.46 | 27.74 | 29.56 |
| CLo, L/h | Mean | 5.78 | 5.59 | 5.88 |
| | SD | 1.80 | 1.74 | 2.07 |

^{a}Harmonic mean and pseudo standard deviation
No statistically significant differences (p ≦ 0.05) were found between tablet and capsule formulations.
Descriptive statistics are presented on non-transformed data. The statistical tests were conducted on the natural logarithmic transformed data for Cmax, AUCT, and AUC and observed (non-transformed data) for λn and CLo. For Tmax, the test was conducted using non-parametric methods.

TABLE 5
Pharmacokinetic Parameter Geometric Mean Ratios and 90% Confidence Intervals for the Efavirenz Tablet Formulation Described in
Table 1 and 2 Herein (4% Croscarmellose Sodium) Relative to the Commercial Efavirenz Capsule Formulation

| | | | Observed Data | | Natural Log-Transformed Data | | | | |
|---|---|---|---|---|---|---|---|---|---|
| Pharmaco- | | | Capsule | Tablet | Capsule | Tablet | | Geometric Mean | 90% CI (% of |
| kinetic | Efavirenz | | (Reference) | (Test) | (Reference) | (Test) | Difference | Ratio (% of | Reference |
| Parameter | Formulation/Dose | N | LSMean | LSMean | LSMean | LSMean | (Test − Reference) | Reference Mean) | Mean) |
| Cmax, μM | Tablet/2 × 300 mg | 21 | 7.49 | 7.61 | 1.954 | 1.989 | 0.034 | 103.48 | 92.73, 115.46 |
| | Tablet/1 × 600 mg | 21 | 7.49 | 7.90 | 1.954 | 2.050 | 0.096 | 110.07 | 98.65, 122.82 |
| AUCT, | Tablet/2 × 300 mg | 21 | 321.10 | 328.37 | 5.720 | 5.743 | 0.024 | 102.38 | 96.02, 109.16 |
| μM-h | Tablet/1 × 600 mg | 21 | 321.10 | 325.35 | 5.720 | 5.739 | 0.019 | 101.94 | 95.61, 108.69 |
| AUC, | Tablet/2 × 300 mg | 21 | 352.95 | 358.42 | 5.819 | 5.836 | 0.017 | 101.76 | 95.95, 107.92 |
| μM-h | Tablet/1 × 600 mg | 21 | 352.95 | 362.16 | 5.819 | 5.848 | 0.029 | 102.98 | 97.11, 109.22 |

The reference and test means are least squares estimated means (LS Mean) from the ANOVA model.
The ratios are the geometric mean ratios for the natural log transformed values.
Formulations were considered bioequivalent if the 90% CI was between 80% and 125%.
CI = confidence interval

Each subject, of a group of healthy (HIV uninfected) adult volunteers, receives a single oral 600 mg dose of the efavirenz commercial capsule (3×200 mg) and two different strengths (2×300 mg or 1×600 mg) of an efavirenz tablet formulation in one of six treatment sequences. There is a minimum 28-day washout period between the administration of each dose. Subjects receive the efavirenz study medication in a fasted state. Blood samples for pharmacokinetic assessments are collected prior to dosing and at 1, 2, 3, 4, 5, 8, 12, 16, 24, 48, 72, 96, 120, 144, 168, 240, 336 and 504 hours after dosing. The pharmacokinetic parameters are calculated based on the individual subject's plasma concentration versus time data.

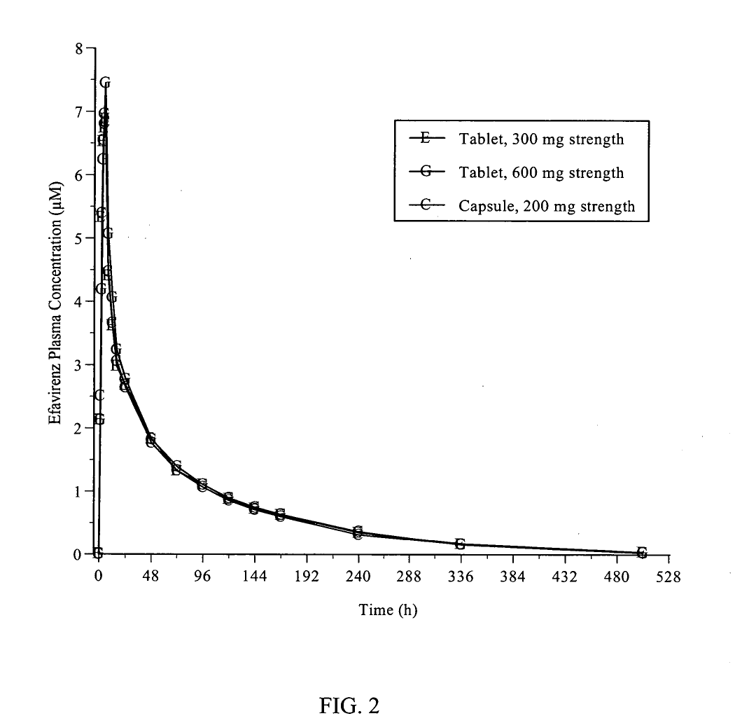

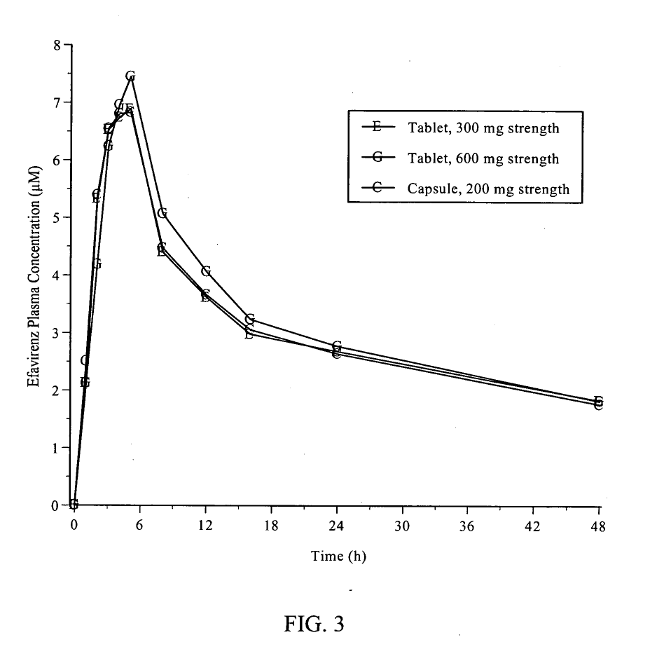

The analytical methodology to measure efavirenz concentrations in plasma may employ reverse-phase high-performance liquid chromatography using ultraviolet detection (HPLC/UV). The assay method may be performed using a liquid-liquid extraction of biological specimens (0.1 mL plasma) spiked with an internal standard. The internal standard is an analog of efavirenz. To each sample, 0.1N NaOH is added, then ethylene dichloride (4.0 mL). The samples are agitated, centrifuged, and the aqueous layer aspirated to waste. The organic layer is evaporated to dryness and then reconstituted with HPLC mobile phase. An aliquot is injected onto the HPLC system. The mean plasma concentration versus time data results obtained during such a study using the 300 mg and 600 mg tablet formulations described in Table 1 and 2 herein, are illustrated in FIGS. 2 and 3.

The tablets of the present invention may be packaged in a container and accompanied by a package insert or label indicating to a user that the efavirenz tablets may be suitable for the treatment of human immunodeficiency virus Type 1 (HIV-1) infection. The package insert or label may also instruct the user on dosage and administration of the tablet composition, for example, 300 mg or 600 mg efavirenz administered orally once daily. The package insert or label may also warn the user of potential side effects, adverse reactions or drug interactions.

One embodiment of the present invention is a pharmaceutical kit comprising at least one efavirenz tablet and a package insert or label indicating to a user that the efavirenz tablet may be suitable for the treatment of human immunodeficiency virus Type 1 (HIV-1) infection. Another embodiment of the present invention is a kit comprising at least one efavirenz tablet and a package insert or label instructing the user on dosage and administration of the tablet composition. Another embodiment of the present invention is a kit comprising at least one efavirenz tablet and a package insert or label warning the user of potential side effects, adverse reactions or drug interactions.

What is claimed is:

1. An efavirenz tablet dosage form, said efavirenz tablet dosage form providing, when administered as a single dose to a subject,
a mean maximum plasma concentration (Cmax) of about 4 μM to about 14 μM;
a mean time of maximum plasma concentration (Tmax) of about 2 hours to about 5 hours; and
a mean area under the plasma concentration versus time curve from time zero to time infinity (AUC) of about 190 μM [symbol] hour to about 470 μM [symbol] hour.

2. A tablet dosage form of claim 1 further having a mean area under the plasma concentration versus time curve from time zero to the last quantifiable concentration-time point (AUCT) of about 180 μM [symbol] hour to about 430 μM [symbol] hour.

3. A tablet dosage form of claim 2 wherein the mean AUCT is about 270 μM [symbol] hour to about 350 μM [symbol] hour.

4. A tablet dosage form of claim 1 further having a mean terminal disposition half-life (T_{1/2}) of about 30 hours to about 140 hours.

5. A tablet dosage form of claim 4 wherein the mean T_{1/2} is about 70 hours to about 100 hours.

6. An efavirenz tablet dosage form, said efavirenz tablet dosage form providing, when administered as a single dose to a subject,
a mean maximum plasma concentration (Cmax) of about 7 μM to about 9 μM;
a mean time of maximum plasma concentration (Tmax) of about 2 hours to about 5 hours; and
a mean area under the plasma concentration versus time curve from time zero to time infinity (AUC) of about 250 μM [symbol] hour to about 400 μM [symbol] hour.

7. A tablet dosage form of claim 1 comprising a therapeutically effective amount of efavirenz and about 4% by weight of a disintegrant relative to the total dry weight of the tablet dosage form.

8. A kit comprising packaging including one or more efavirenz tablet of claim 1 and a package insert or label indicating to a user that the efavirenz tablet may be suitable for the treatment of human immunodeficiency virus Type 1 (HIV-1) infection.

9. A tablet dosage form of claims 1, 2, 3, 4, 5, 6 or 7 comprising 300 mg of efavirenz per tablet.

10. A tablet dosage form of claims 1, 2, 3, 4, 5, 6 or 7 comprising 600 mg of efavirenz per tablet.

11. A kit comprising at least one efavirenz tablet of claim 1 and a package insert or label instructing the user on dosage and administration of the tablet dosage form.

12. A kit comprising at least one efavirenz tablet of claim 1 and a package insert or label warning the user of potential side effects, adverse reactions or drug interactions.

13. A method of treating human immunodeficiency virus Type 1 (HIV-1) infection comprising administering to a mammal the efavirenz tablet formulation of claim 1.

14. A method of treating human immunodeficiency virus Type 1 (HIV-1) infection comprising administering to a mammal an efavirenz tablet dosage form, said efavirenz tablet dosage form providing, when administered as a single dose to a subject,
a mean maximum plasma concentration (Cmax) of about 4 μM to about 14 μM;
a mean time of maximum plasma concentration (Tmax) of about 2 hours to about 5 hours; and
a mean area under the plasma concentration versus time curve from time zero to time infinity (AUC) of about 190 μM [symbol] hour to about 470 μM [symbol] hour.

15. A 300 mg efavirenz tablet dosage form suitable for use in treating human immunodeficiency virus Type 1 (HIV-1) infection, said 300 mg efavirenz tablet dosage form providing,
an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows:
(a) between about 62% and about 75% of the efavirenz tablet dosage form is dissolved after about 10 minutes in the type II dissolution apparatus,
(b) between about 90% and about 95% of the efavirenz tablet dosage form is dissolved after about 20 minutes in the type II dissolution apparatus,
(c) between about 96% and about 98% of the efavirenz tablet dosage form is dissolved after about 30 minutes in the type II dissolution apparatus, and
(d) between about 99% and about 100% of the efavirenz tablet dosage form is dissolved after about 45 minutes in the type II dissolution apparatus.

16. The 300 mg efavirenz tablet dosage form of claim 15 wherein 100% of the 300 mg efavirenz tablet dosage form is dissolved after about 45 minutes.

17. A 300 mg efavirenz tablet dosage form suitable for use in treating human immunodeficiency virus Type 1 (HIV-1) infection, said 300 mg efavirenz tablet dosage form providing,
an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows:
(a) between about 62% and about 75% of the efavirenz tablet dosage form is dissolved after about 10 minutes in the type II dissolution apparatus,
(b) between about 89% and about 95% of the efavirenz tablet dosage form is dissolved after about 20 minutes in the type II dissolution apparatus,
(c) between about 94% and about 98% of the efavirenz tablet dosage form is dissolved after about 30 minutes in the type II dissolution apparatus, and
(d) between about 97% and about 100% of the efavirenz tablet dosage form is dissolved after about 45 minutes in the type II dissolution apparatus.

18. The 300 mg efavirenz tablet dosage form of claim 17 wherein 100% of the 300 mg efavirenz tablet dosage form is dissolved after about 45 minutes.

19. A 600 mg efavirenz tablet dosage form suitable for use in treating human immunodeficiency virus Type 1 (HIV-1) infection, said 600 mg efavirenz tablet dosage form providing,
an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows:
(a) between about 49% and about 71% of the efavirenz tablet dosage form is dissolved after about 10 minutes in the type II dissolution apparatus,
(b) between about 87% and about 95% of the efavirenz tablet dosage form is dissolved after about 20 minutes in the type II dissolution apparatus,
(c) between about 97% and about 99% of the efavirenz tablet dosage form is dissolved after about 30 minutes in the type II dissolution apparatus, and
(d) between about 99% and about 100% of the efavirenz tablet dosage form is dissolved after about 45 minutes in the type II dissolution apparatus.

20. The 600 mg efavirenz tablet dosage form of claim 19 wherein 100% of the 600 mg efavirenz tablet dosage form is dissolved after about 45 minutes.

21. A 600 mg efavirenz tablet dosage form suitable for use in treating human immunodeficiency virus Type 1 (HIV-1) infection, said 600 mg efavirenz tablet dosage form providing,
an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows:
(a) between about 49% and about 71% of the efavirenz tablet dosage form is dissolved after about 10 minutes in the type II dissolution apparatus,
(b) between about 86% and about 95% of the efavirenz tablet dosage form is dissolved after about 20 minutes in the type II dissolution apparatus,
(c) between about 94% and about 99% of the efavirenz tablet dosage form is dissolved after about 30 minutes in the type II dissolution apparatus, and
(d) between about 97% and about 100% of the efavirenz tablet dosage form is dissolved after about 45 minutes in the type II dissolution apparatus.

22. The 600 mg efavirenz tablet dosage form of claim 21 wherein 100% of the 600 mg efavirenz tablet dosage form is dissolved after about 45 minutes.

23. A method of treating human immunodeficiency virus Type 1 (HIV-1) infection comprising administering to a mammal a 300 mg efavirenz tablet dosage form, said 300 mg efavirenz tablet dosage form providing,
an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows:
(a) between about 62% and about 75% of the efavirenz tablet dosage form is dissolved after about 10 minutes in the type II dissolution apparatus,
(b) between about 90% and about 95% of the efavirenz tablet dosage form is dissolved after about 20 minutes in the type II dissolution apparatus,
(c) between about 96% and about 98% of the efavirenz tablet dosage form is dissolved after about 30 minutes in the type II dissolution apparatus, and
(d) between about 99% and about 100% of the efavirenz tablet dosage form is dissolved after about 45 minutes in the type II dissolution apparatus.

24. The method of claim 23 wherein 100% of the 300 mg efavirenz tablet dosage form is dissolved after about 45 minutes.

25. A method of treating human immunodeficiency virus Type 1 (HIV-1) infection comprising administering to a mammal a 300 mg efavirenz tablet dosage form, said 300 mg efavirenz tablet dosage form providing,
an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows:
(a) between about 62% and about 75% of the efavirenz tablet dosage form is dissolved after about 10 minutes in the type II dissolution apparatus,
(b) between about 89% and about 95% of the efavirenz tablet dosage form is dissolved after about 20 minutes in the type II dissolution apparatus,
(c) between about 94% and about 98% of the efavirenz tablet dosage form is dissolved after about 30 minutes in the type II dissolution apparatus, and
(d) between about 97% and about 100% of the efavirenz tablet dosage form is dissolved after about 45 minutes in the type II dissolution apparatus.

26. The method of claim 25 wherein 100% of the 300 mg efavirenz tablet dosage form is dissolved after about 45 minutes.

27. A method of treating human immunodeficiency virus Type 1 (HIV-1) infection comprising administering to a mammal a 600 mg efavirenz tablet dosage form, said 600 mg efavirenz tablet dosage form providing,
an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows:
(a) between about 49% and about 71% of the efavirenz tablet dosage form is dissolved after about 10 minutes in the type II dissolution apparatus,
(b) between about 87% and about 95% of the efavirenz tablet dosage form is dissolved after about 20 minutes in the type II dissolution apparatus,
(c) between about 97% and about 99% of the efavirenz tablet dosage form is dissolved after about 30 minutes in the type II dissolution apparatus, and
(d) between about 99% and about 100% of the efavirenz tablet dosage form is dissolved after about 45 minutes in the type II dissolution apparatus.

28. The method of claim 27 wherein 100% of the 600 mg efavirenz tablet dosage form is dissolved after about 45 minutes.

29. A method of treating human immunodeficiency virus Type 1 (HIV-1) infection comprising administering to a mammal a 600 mg efavirenz tablet dosage form, said 600 mg efavirenz tablet dosage form providing,
an in vitro dissolution profile, when measured in a type II dissolution apparatus, according to U.S. Pharmacopeia XXIV, at about 37° C. in 2% (w/v) aqueous sodium lauryl sulfate at about 50 rpm, as follows:
(a) between about 49% and about 71% of the efavirenz tablet dosage form is dissolved after about 10 minutes in the type II dissolution apparatus,
(b) between about 86% and about 95% of the efavirenz tablet dosage form is dissolved after about 20 minutes in the type II dissolution apparatus,
(c) between about 94% and about 99% of the efavirenz tablet dosage form is dissolved after about 30 minutes in the type II dissolution apparatus, and
(d) between about 97% and about 100% of the efavirenz tablet dosage form is dissolved after about 45 minutes in the type II dissolution apparatus.

30. The method of claim 29 wherein 100% of the 600 mg efavirenz tablet dosage form is dissolved after about 45 minutes.
